(19) United States
(12) Patent Application Publication
(10) Pub. No.: US 2023/0331794 A1
(43) Pub. Date: Oct. 19, 2023

(54) PEPTIDE FOR PREVENTING OR TREATING NEURODEGENERATIVE DISEASES AND PHARMACEUTICAL COMPOSITION COMPRISING SAME

(71) Applicant: SEOUL NATIONAL UNIVERSITY R&DB FOUNDATION, Seoul (KR)

(72) Inventors: Nam Ki LEE, Seoul (KR); Gyeongji YOO, Seoul (KR)

(73) Assignee: SEOUL NATIONAL UNIVERSITY R&DB FOUNDATION, Seoul (KR)

(21) Appl. No.: 18/028,932

(22) PCT Filed: Sep. 27, 2021
(86) PCT No.: PCT/KR2021/013121

§ 371 Date: Mar. 28, 2023

(30) Foreign Application Priority Data
Sep. 29, 2020 (KR) 10-2020-0126651

(51) Int. Cl. C07K 14/47; A61P 25/28

(52) U.S. Cl. CPC C07K 14/47; A61P 25/28; A61K 38/00

(57) ABSTRACT

The present invention relates to a peptide which can prevent and treat a neurodegenerative disease and a pharmaceutical composition comprising the peptide. The peptide of the present invention inhibits the binding of v-SNARE protein and alpha-synuclein oligomers, thus lowering neural toxicity caused by the alpha-synuclein oligomers and allowing effective use in the development of therapeutic agents for a neurodegenerative disease such as Parkinson’s disease.

### TECHNICAL FIELD

The present invention relates to a peptide which can prevent and treat a neurodegenerative disease by lowering neural toxicity caused by alpha-synuclein oligomers. Specifically, the present invention relates to a peptide which exhibits preventive and therapeutic effects on a neurodegenerative disease such as Parkinson’s disease by inhibiting the binding of v-SNARE protein and alpha-synuclein oligomers and a pharmaceutical composition comprising the same.

### BACKGROUND ART

Neurodegenerative diseases collectively refer to diseases caused by the death of nerve cells that slowly and continuously progress in one part or several parts of the nervous system. Among them, Parkinson’s disease, in particular, is the second most prevalent neurodegenerative disease after Alzheimer’s disease, and occurs at a rate of about 1 to 2 per 1,000 people aged 60 years or older. Typical symptoms may include bradykinesia (slow movement), tremor at rest, muscle rigidity, and postural instability, etc.

Parkinson’s disease is a disease in which nerve cells that secrete dopamine in the substantia nigra located in the midbrain gradually disappear due to unknown causes. Dopamine is used when stimulating or inhibiting motor nerves, and plays a role in controlling humans to move normally through these functions. When dopamine is produced in a nerve cell, it is transported to the end of the nerve cell by a transporter called a synaptic vesicle and stored therein. In this case, the SNARE protein plays a key role in ensuring that the information contained in the vesicle is not lost and accurately reaches and fuses with the target organ.

SNARE proteins are classified into two types, t-SNARE and v-SNARE, depending on their location and shape. t-SNARE is a complex composed of proteins syntaxin-1A and SNAP-25, and is present in the plasma membrane of nerve cells. v-SNARE is a protein called synaptobrevin-2 or VAMP-2 (vesicle associated membrane protein), which is a membrane protein present in the membrane of nerve vesicles. The t-SNARE protein and the v-SNARE protein form a 4-helical complex with each other, thereby inducing the distance between the membrane of the nerve vesicle and the plasma membrane of the cell to be close, and thereby allowing fusion of the nerve vesicle and neurotransmission to occur.

Many studies are being conducted on the detailed mechanism of action and application of the SNARE protein as such a protein complex, and the major factor in Parkinson’s disease known to date is alpha-synuclein oligomers. However, the detailed mechanisms of how abnormal oligomers of alpha-synuclein protein and genetic mutations induce neural toxicity are still unclear.

Antibodies for inhibiting alpha-synuclein oligomers are being developed (Korean Patent Application Publication No. 10-2009-0041066, etc.), but these antibodies also bind to normal alpha-synuclein single bodies, are not specific to the oligomers, and have the low permeability to the blood-brain barrier. Therefore, there is a limit to the therapeutic efficacy.

Under this background, the present inventors have found that alpha-synuclein oligomers exhibit neural toxicity by binding to the v-SNARE protein, which is essential in the neurotransmission process. In addition, the present inventors have discovered a novel peptide that inhibits the binding of alpha-synuclein oligomers and v-SNARE protein to lower neural toxicity and has the high permeability to the blood-brain barrier. Based on the above, the present inventors completed the present invention.

### PRIOR ART DOCUMENT

### Patent Document

(Patent Document 1) Korean Patent Application Publication No. 10-2009-0041066

### DETAILED DESCRIPTION OF INVENTION

### Technical Problem

An object of the present invention is to confirm the mechanism by which alpha-synuclein oligomers act on v-SNARE protein to induce neural toxicity, and to obtain a novel substance that lowers neural toxicity.

In addition, another object of the present invention is to provide a peptide which exhibits preventive and therapeutic effects on a neurodegenerative disease such as Parkinson’s disease by inhibiting the binding of v-SNARE protein and alpha-synuclein oligomers and a pharmaceutical composition comprising the same.

### Solution to Problem

In order to achieve the above object, the present invention provides a peptide composed of the entire amino acid sequence of SEQ ID NO: 1 or a part thereof.

In addition, the present invention provides a peptide comprising the amino acid sequence of SEQ ID NO: 2.

In addition, the present invention provides a peptide comprising the amino acid sequence of SEQ ID NO: 3.

In addition, the present invention provides a peptide comprising the amino acid sequence of SEQ ID NO: 4.

In one embodiment, the peptide is characterized in that it is for preventing or treating a neurodegenerative disease.

In one embodiment, the neurodegenerative disease may be at least one selected from the group consisting of Parkinson’s disease, Alzheimer’s disease, Pick’s disease, Huntington’s disease, Lou Gehrig’s disease, prion disease, Lewy body dementia, multiple system atrophy, progressive supranuclear palsy, Friedreich’s ataxia, temporal lobe epilepsy, and stroke.

In one embodiment, the peptide may inhibit the binding of alpha-synuclein oligomers and v-SNARE protein.

The present invention provides a pharmaceutical composition for preventing or treating a neurodegenerative disease, comprising a peptide composed of the entire amino acid sequence of SEQ ID NO: 1 or a part thereof.

In addition, the present invention provides a pharmaceutical composition for preventing or treating a neurodegenerative disease, comprising a peptide comprising the amino acid sequence of SEQ ID NO: 2, SEQ ID NO: 3 or SEQ ID NO: 4.

The present invention provides a method for preventing or treating a neurodegenerative disease by administering the entire amino acid sequence of SEQ ID NO: 1 or a part thereof to a patient with a neurodegenerative disease.

In addition, the present invention provides a method for preventing or treating a neurodegenerative disease by administering a peptide comprising the amino acid sequence of SEQ ID NO: 2, SEQ ID NO: 3 or SEQ ID NO: 4 to a patient with a neurodegenerative disease.

### Effects of Invention

The peptide of the present invention inhibits the binding of v-SNARE protein and alpha-synuclein oligomers, thus lowering neural toxicity caused by the alpha-synuclein oligomers and allowing effective use in the development of therapeutic agents for a neurodegenerative disease such as Parkinson’s disease.

### BRIEF DESCRIPTION OF DRAWINGS

FIGS. 1a and 1b illustrate the results obtained by confirming that whether alpha-synuclein oligomers and monomers inhibit SNARE protein-mediated vesicle membrane fusion through fluorescence resonance energy transfer (FRET).

FIGS. 2a to 2d illustrate the results obtained by observing the effect of alpha-synuclein monomers and/or oligomers on the clustering phenomenon of v-SNARE vesicles.

FIGS. 3a and 3b illustrate the results obtained by confirming that whether alpha-synuclein monomer mutant T44P/A89P interferes with the binding between alpha-synuclein oligomers and v-SNARE, thereby alleviating the inhibition of vesicle membrane fusion of alpha-synuclein oligomers.

FIGS. 4a and 4b illustrate the results obtained by confirming that whether the inhibition of vesicle membrane fusion of alpha-synuclein oligomers is alleviated because the binding between the oligomers and v-SNARE is interfered in the vesicle in which PS (Phosphatidyl Serine) having a negative charge has been removed from the v-SNARE lipid membrane.

FIGS. 5a and 5b illustrate the results obtained by observing that the clustering of v-SNARE vesicles was reduced when alpha-synuclein monomer mutant T44P/A89P interfered with the binding between alpha-synuclein oligomers and v-SNARE.

FIG. 6a is a schematic diagram showing the peptide of the present invention derived from the C-terminal region of alpha-synuclein (amino acid sequence 96-140) and adjacent amino acids (amino acid sequence 81-95).

FIG. 6b illustrates the results obtained by observing the degree of recovery of vesicle fusion by treatment with the peptides of SEQ ID NOs: 2 to 4 together with 10 nM alpha-synuclein oligomers.

FIG. 6c illustrates the results obtained by observing the degree of recovery of vesicle fusion when treated with the peptides of SEQ ID NOs: 2 to 4 alone without alpha-synuclein oligomers.

FIG. 6d illustrates the results obtained by confirming that there was no significant difference in vesicle fusion when additionally treated with vesicle (F) without SNARE protein.

### BEST MODE FOR CARRYING OUT THE INVENTION

Hereinafter, with reference to the accompanying drawings, embodiments and examples of the present invention will be described in detail so that those of ordinary skill in the art to which the present invention belongs can easily practice the present invention. However, the present invention may be implemented in various forms and is not limited to the embodiments and examples described herein.

Throughout the present specification, when a certain part “includes” a certain component, it means that other components may be further included, rather than excluding other components, unless otherwise stated.

The present invention relates to a peptide having a preventive or therapeutic effect on a neurodegenerative disease and a pharmaceutical composition comprising the same.

Alpha-synuclein is a major component of Lewy body, which is a typical pathological feature of several neurodegenerative diseases. Under pathological conditions, the detailed mechanisms by which these oligomers destroy nerve cells have not yet been fully elucidated.

The t-SNARE protein present in the plasma membrane of nerve cells and the v-SNARE protein present in the membrane of nerve vesicles form a 4-helical SNARE complex with each other, thereby inducing the distance between the membrane of the nerve vesicle and the plasma membrane of the cell to be close, and thereby allowing fusion of the nerve vesicle and neurotransmission to occur.

In the present invention, it was found that alpha-synuclein oligomers inhibit vesicle fusion by SNARE complex formation even in small amounts, whereas normal alpha-synuclein monomers do not interfere with vesicle fusion in the absence of oligomers.

Specifically, it was found that when alpha-synuclein monomers were mixed with the vesicles comprising v-SNARE and observed, the vesicles were present in an appropriately spread state, but when alpha-synuclein oligomers were added, clusters in which several vesicles were united were observed. In other words, alpha-synuclein oligomers bind to v-SNARE protein to cluster nerve vesicles, and cannot bind to t-SNARE in the cell membrane at the end of nerve cells to inhibit the vesicle fusion process that should occur later, and neurotransmission process (dopamine release action) does not occur.

From the above experimental results, it can be seen that a substance that inhibits the interaction between alpha-synuclein oligomers and v-SNARE protein can be used as a therapeutic agent for Parkinson’s disease. Therefore, the present inventors have invented specific blocking peptides that inhibit the binding between alpha-synuclein oligomers and v-SNARE protein by using the C-terminal region of alpha-synuclein (amino acid sequence 96-140) and a part of adjacent amino acid (amino acid sequence 81-95) sequence.

As used herein, the term “neurodegenerative” includes all of the transition of nerve cells from a normal state to a state with reduced function, a genetic decline in function, and a sporadic decline in function, and includes the slow and uninterrupted death of nerve cells in one or several parts of the nervous system.

As used herein, the term “prevention” refers to any action of inhibiting or delaying the onset of a neurodegenerative disease by administration of the pharmaceutical composition according to the present invention, and “treatment” refers to any action in which symptoms of a subject suspected of and suffering from a neurodegenerative disease are improved or beneficially changed by administration of the pharmaceutical composition.

The pharmaceutical composition of the present invention can be formulated into powders, granules, tablets, coated tablets, pills, dragees, capsules, solutions, suspended solutions, gels, syrups, slurries, suppositories, enemas, emulsions, pastes, ointments, creams, lotions, powders, sprays or suspensions.

The pharmaceutical composition of the present invention may further comprise appropriate carriers, excipients or diluents commonly used in the preparation of pharmaceutical compositions. For example, they include lactose, dextrose, sucrose, sorbitol, mannitol, xylitol, erythritol, mannitol, starch, gum acacia, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate or mineral oil, etc.

Hereinafter, the present invention will be described in more detail through the examples, but the following examples are for illustrative purposes only and are not intended to limit the scope of the present invention.

### [Example 1] Confirmation of SNARE Protein-Mediated Vesicle Membrane Fusion Inhibitory Effect by Alpha-Synuclein Oligomers

Artificial vesicles were prepared with a composition of 71% PC (Phosphatidyl Choline), 20% cholesterol, 7% PS (Phosphatidyl Serine), and 2% fluorescent dye, and t-SNARE and v-SNARE proteins were added to the membrane of the vesicle to construct two types of vesicles, respectively. Different fluorescent dyes (DiI and DiD) were added to the two types of vesicles so that Fluorescence Resonance Energy Transfer (FRET) could occur when the two types of vesicles came closer to each other at a distance of 10 nm or less. That is, when the vesicles comprising the t-SNARE protein and the vesicles comprising the v-SNARE protein come closer to each other at a distance of 10 nm or less, and the lipid membranes are mixed, the fluorescent dye is also mixed, resulting in fluorescence according to FRET, thereby confirming membrane fusion.

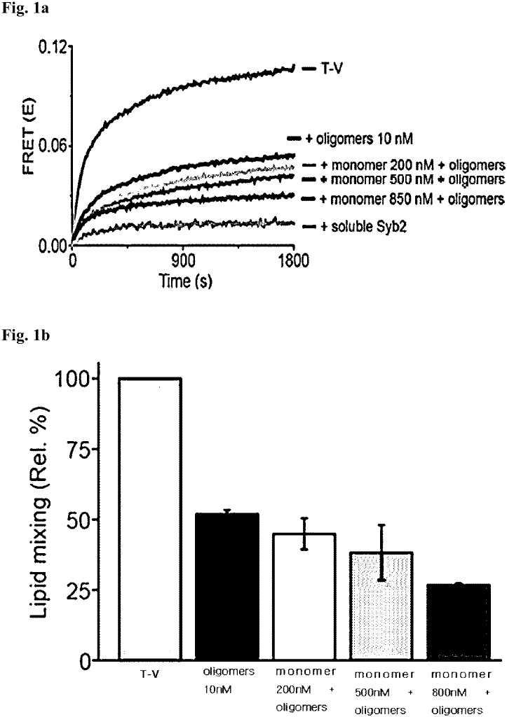

In order to confirm that alpha-synuclein oligomers and monomers inhibit SNARE protein-mediated vesicle membrane fusion, observation using FRET was performed under the following conditions. The FRET signal change between the two types of vesicles over time are shown in FIG. 1a, and the relative degree of vesicle fusion is shown in FIG. 1b.

When the two types of vesicles are mixed (lipid concentration of 10 ₁₁M each) and irradiated with light at a wavelength of 560 nm, the two SNARE proteins meet to form a complex and the lipid membrane is fused, which can be confirmed by observing the fluorescence intensity at a wavelength of 690 nm. The degree of vesicle fusion in the case where it was not treated with alpha-synuclein monomers or oligomers (T-V in FIG. 1) was set as the reference (100%) (FIG. 1b).

It was confirmed that when 10 nM alpha-synuclein oligomers were incubated in v-SNARE vesicles for 10 minutes and then t-SNARE vesicles were mixed, the degree of lipid membrane fusion was reduced by about 50% due to alpha-synuclein oligomers.

In addition, it was confirmed that when 200 nM, 500 nM, and 850 nM of alpha-synuclein monomers were mixed with v-SNARE vesicles and incubated for 10 minutes and then 10 nM alpha-synuclein oligomers and t-SNARE vesicles were added, the effect of the oligomers to inhibit SNARE protein-mediated lipid membrane fusion was further increased as the concentration of the monomers was increased.

From the above results, it can be seen that alpha-synuclein oligomers inhibit SNARE protein-mediated vesicle membrane fusion even in small amounts, and thus the pathological effect of inhibiting neurotransmission in this way is further increased depending on the concentration of the monomer when alpha-synuclein monomers are present.

### [Example 2] Confirmation of Clustering of v-SNARE Vesicles by Alpha-Synuclein Oligomers

In order to confirm clustering of v-SNARE vesicles by alpha-synuclein oligomers, a mixture of vesicles and alpha-synuclein was observed under a cryo-electron microscope.

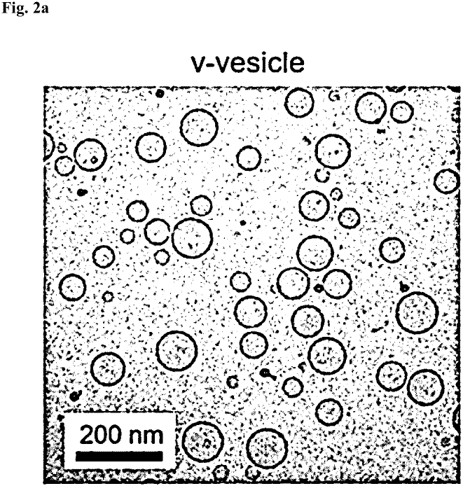

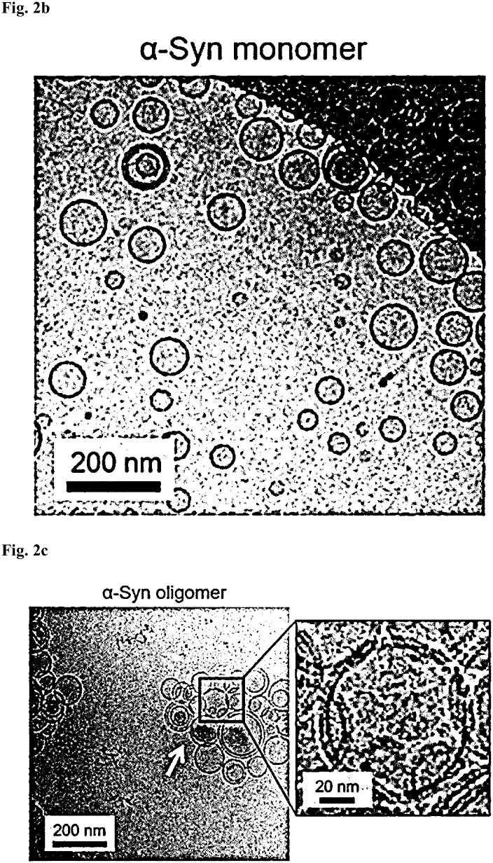

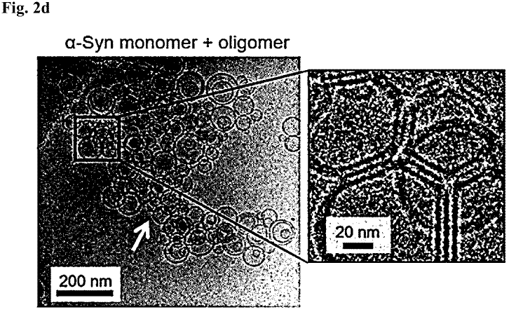

When only v-SNARE vesicles were observed alone, each vesicle was spread at a certain distance (FIG. 2a), and even when alpha-synuclein monomers were mixed, v-SNARE vesicles were spread appropriately (FIG. 2b). However, it was confirmed that when alpha-synuclein oligomers were mixed, a cluster of several vesicles was observed (FIG. 2c), and when alpha-synuclein monomers and oligomers were added to v-SNARE vesicles, a greater number of vesicles were clustered and the membrane between vesicles was more tightly adhered than when oligomers alone were added (FIG. 2d).

In other words, alpha-synuclein oligomers bind to v-SNARE and make nerve vesicles cluster, preventing them from binding to t-SNARE at the end of nerve cells, thereby inhibiting vesicle fusion and neurotransmission. It was confirmed that this phenomenon was further increased when alpha-synuclein monomers were present together.

### [Example 3] Confirmation of SNARE Protein-Mediated Vesicle Membrane Fusion Inhibitory Effect when Interfering with Binding Between Alpha-Synuclein Oligomers and v-SNARE

It was confirmed that alpha-synuclein oligomers clustered v-SNARE vesicles and interfered with binding to t-SNARE vesicles that could occur later. Therefore, whether the above neurotoxic effect was reduced when the binding between alpha-synuclein oligomers and v-SNARE was prevented was confirmed through FRET observation as in Example 1, as follows.

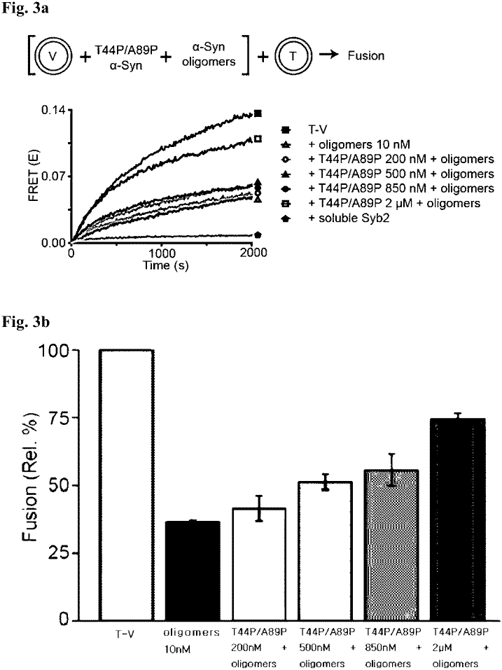

An alpha-synuclein monomer mutant T44P/A89P, which could not bind to the lipid membrane but could bind to the v-SNARE protein, was prepared, and a lipid membrane fusion experiment was performed at the in vitro level. As a result, T44P/A89P competitively interfered with the binding of alpha-synuclein oligomers and v-SNARE, thereby reducing the effect of alpha-synuclein oligomers and recovering the degree of lipid membrane fusion (FIGS. 3a and 3b).

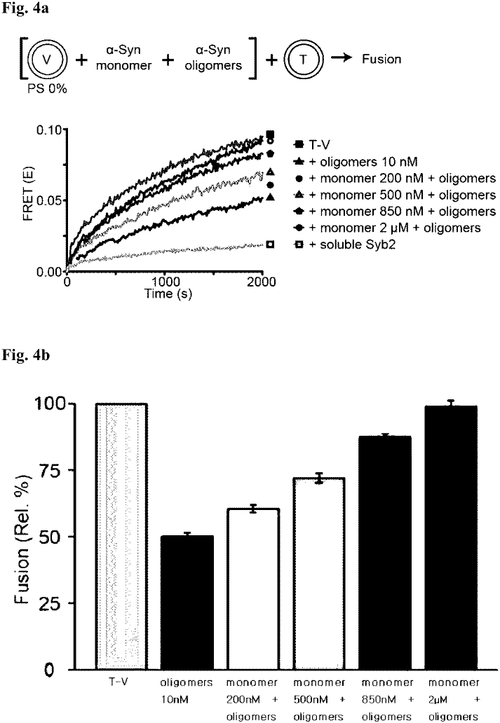

In addition, the vesicles were prepared in which PS (Phosphatidyl Serine) having a negative charge has been removed from the v-SNARE lipid membrane so that alpha-synuclein could not bind, and the v-SNARE vesicles were treated with alpha-synuclein monomers and oligomers. As a result, similarly, the effect of alpha-synuclein oligomers was reduced, and the degree of lipid membrane fusion was recovered (FIGS. 4a and 4b).

In other words, it was confirmed that the pathological effect of the alpha-synuclein oligomers can be reduced when the binding between v-SNARE and alpha-synuclein oligomers is inhibited.

### [Example 4] Confirmation of Reduction of Vesicle Clustering Effect of Alpha-Synuclein Oligomers by T44P/A89P

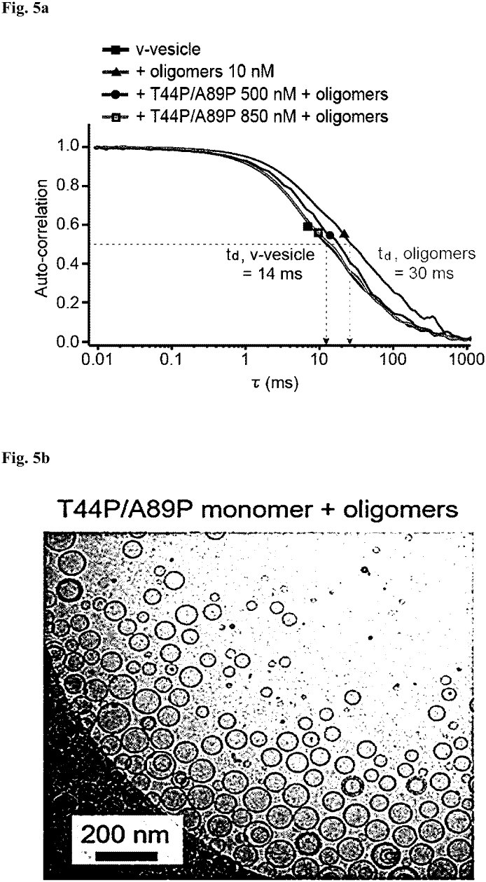

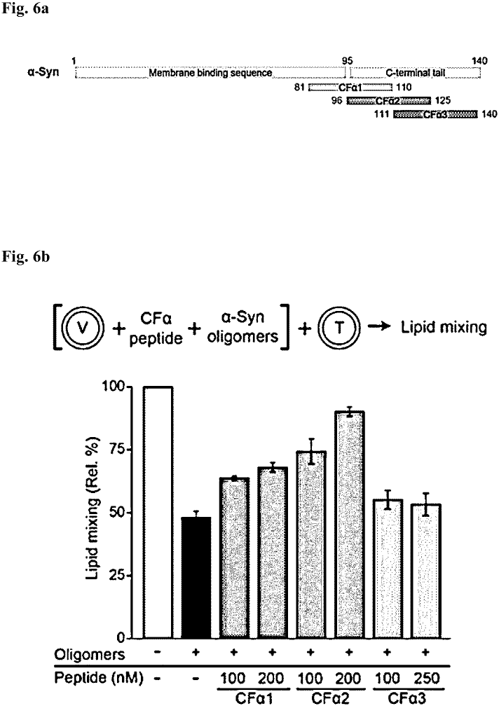

Fluorescence correlation spectroscopy experiment was performed in order to confirm that whether the effect of alpha-synuclein oligomers to cluster vesicles is also reduced by the alpha-synuclein monomer mutant T44P/A89P, which can bind to v-SNARE and cannot bind to the lipid membrane of the vesicles, and the shape of the vesicles was observed under a cryo-electron microscope. As shown in FIG. 5a, in v-SNARE vesicles mixed with 10 nM alpha-synuclein oligomers, the size of moving units was increased due to clustering, and as a result, the diffusion time (30 ms) was increased by about two times compared to that of the vesicle alone measurement group (14 ms). On the other hand, when T44P/A89P was incubated with v-SNARE vesicles together with alpha-synuclein oligomers, the clustering of the vesicles was reduced, and the diffusion time was recovered to a level similar to that when the vesicles were measured alone.

When T44P/A89P and alpha-synuclein oligomers were mixed with the v-SNARE vesicles, it was confirmed through a cryo-electron microscope that the clustering seen in Example 2 disappeared and the spaces were separated little by little as if there was a repulsive force between the vesicles (FIG. 5b). The above results show that a substance capable of binding to v-SNARE and thus competitively inhibiting the binding of alpha-synuclein oligomers has a therapeutic effect of inhibiting the toxicity of alpha-synuclein oligomers.

### [Example 5] Confirmation of Synthetic Peptides That Interfere with the Interaction Between Alpha-Synuclein Oligomers and v-SNARE

The purpose of this example was to prepare a small peptide inhibitor that competes with alpha-synuclein oligomers for v-SNARE binding sites and consequently interferes with the binding of the oligomers and the SNARE protein.

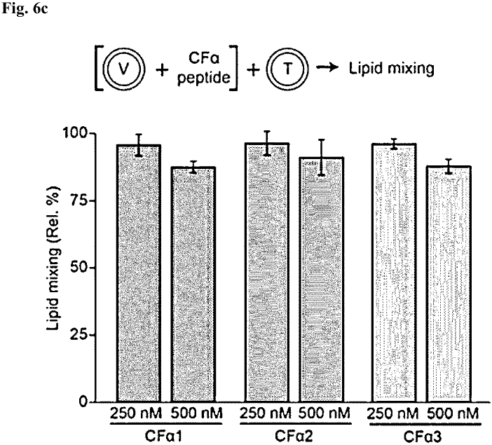

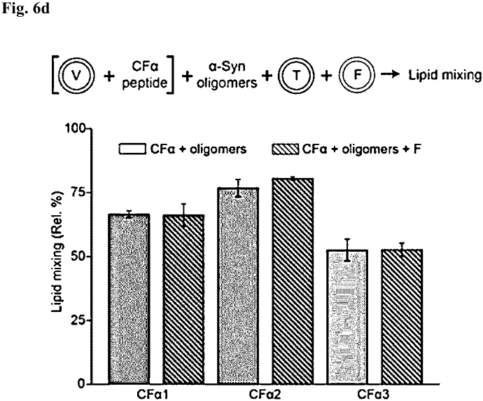

As shown in FIG. 6a and Table 1, a small synthetic peptide that blocks the binding of v-SNARE and alpha-synuclein oligomers was designed using a part of SEQ ID NO: 1 comprising the C-terminal region of alpha-synuclein (amino acid sequence 96-140) and adjacent amino acids.

TABLE 1

| | | |
|---|---|---|
| NO. | Position | Sequence |
| SEQ ID NO: 1 | α-Syn 81-140 | TVEGAGSIAAATGFVKKDQLGKNEEGAPQE GILEDMPVDPDNEAYEMPSEEGYQDYEPEA |
| SEQ ID NO: 2(CFα1) | α-Syn 81-110 | TVEGAGSIAAATGFVKKDQLGKNEEGAPQE |
| SEQ ID NO: 3(CFα2) | α-Syn 96-125 | KKDQLGKNEEGAPQEGILEDMPVDPDNEAY |
| SEQ ID NO: 4(CFα3) | α-Syn 111-140 | GILEDMPVDPDNEAYEMPSEEGYQDYEPEA |

Whether the peptides of SEQ ID NO: 2 (CFα1), SEQ ID NO: 3 (CFα2) and SEQ ID NO: 4 (CFα3) inhibit the binding between alpha-synuclein oligomers and v-SNARE was confirmed through FRET observation as in Example 1.

As shown in FIG. 6b, vesicle fusion was inhibited when treated with 10 nM alpha-synuclein oligomers, whereas vesicle fusion was recovered when treated together with the peptides of SEQ ID NOs: 2 to 4. In particular, the peptide of SEQ ID NO: 3 most efficiently inhibited the binding of v-SNARE and alpha-synuclein oligomers and recovered the fusion process of the vesicles.

On the other hand, as shown in FIG. 6c, the peptide alone had little effect on the vesicle fusion process in the absence of alpha-synuclein oligomers. In addition, as shown in FIG. 6d, as a result of comparison with the case where vesicles (F) without SNARE protein were additionally added, there was no significant difference in vesicle fusion, indicating that the peptides of SEQ ID NOs: 2 to 4 bind to the v-SNARE protein, not to the lipid membrane of the vesicle.

From the above results, it was confirmed that the peptide of the present invention can reduce the pathological effect of inhibiting neurotransmission by alleviating the effect of alpha-synuclein oligomers inhibiting vesicle fusion by binding to v-SNARE protein.

1. A peptide composed of the entire amino acid sequence of SEQ ID NO: 1 or a part thereof.

2. The peptide according to claim 1, wherein the part thereof has the amino acid sequence of SEQ ID NO: 2.

3. The peptide according to claim 1, wherein the part thereof has the amino acid sequence of SEQ ID NO: 3.

4. The peptide according to claim 1, wherein the part thereof has the amino acid sequence of SEQ ID NO: 4.

5. The peptide according to claim 1, wherein the peptide is for preventing or treating a neurodegenerative disease.

6. The peptide for preventing or treating a neurodegenerative disease according to claim 5, wherein the neurodegenerative disease is at least one selected from the group consisting of Parkinson’s disease, Alzheimer’s disease, Pick’s disease, Huntington’s disease, Lou Gehrig’s disease, prion disease, Lewy body dementia, multiple system atrophy, progressive supranuclear palsy, Friedreich’s ataxia, temporal lobe epilepsy, and stroke.

7. The peptide for preventing or treating a neurodegenerative disease according to claim 6, wherein the neurodegenerative disease is Parkinson’s disease.

8. The peptide according to claim 1, wherein the peptide inhibits the binding of alpha-synuclein oligomers and v-SNARE protein.

9. A pharmaceutical composition for preventing or treating a neurodegenerative disease, comprising the peptide according to claim 1.

10. The pharmaceutical composition for preventing or treating a neurodegenerative disease according to claim 9, wherein the neurodegenerative disease is at least one selected from the group consisting of Parkinson’s disease, Alzheimer’s disease, Pick’s disease, Huntington’s disease, Lou Gehrig’s disease, prion disease, Lewy body dementia, multiple system atrophy, progressive supranuclear palsy, Friedreich’s ataxia, temporal lobe epilepsy, and stroke.

11. The pharmaceutical composition for preventing or treating a neurodegenerative disease according to claim 10, wherein the neurodegenerative disease is Parkinson’s disease.

12. The pharmaceutical composition for preventing or treating a neurodegenerative disease according to claim 9, wherein the peptide inhibits the binding of alpha-synuclein oligomers and v-SNARE protein.

13. A method for treating a neurodegenerative disease, the method comprising administering a composition according to claim 1 to a subject in need thereof.

14. The method according to claim 13, wherein the neurodegenerative disease is at least one selected from the group consisting of Parkinson’s disease, Alzheimer’s disease, Pick’s disease, Huntington’s disease, Lou Gehrig’s disease, prion disease, Lewy body dementia, multiple system atrophy, progressive supranuclear palsy, Friedreich’s ataxia, temporal lobe epilepsy, and stroke.

15. The method according to claim 13, wherein the neurodegenerative disease is Parkinson’s disease.

16. The method according to claim 13, wherein the subject requires inhibiting the binding of alpha-synuclein oligomers and v-SNARE protein.

17. The method according to claim 13, wherein the part thereof has the amino acid sequence of SEQ ID NO: 2.

18. The method according to claim 13, wherein the part thereof has the amino acid sequence of SEQ ID NO: 3.

19. The method according to claim 13, wherein the part thereof has the amino acid sequence of SEQ ID NO: 4.


SEQUENCE LISTING (4 sequences)

SEQ ID NO: 1 - protein, 60 aa, Artificial Sequence
  alpha-Syn 81-140
TVEGAGSIAAATGFVKKDQLGKNEEGAPQEGILEDMPVDPDNEAYEMPSEEGYQDYEPEA

SEQ ID NO: 2 - protein, 30 aa, Artificial Sequence
  alpha-Syn 81-110
TVEGAGSIAAATGFVKKDQLGKNEEGAPQE

SEQ ID NO: 3 - protein, 30 aa, Artificial Sequence
  alpha-Syn 96-125
KKDQLGKNEEGAPQEGILEDMPVDPDNEAY

SEQ ID NO: 4 - protein, 30 aa, Artificial Sequence
  alpha-Syn 111-140
GILEDMPVDPDNEAYEMPSEEGYQDYEPEA